(19) United States
(12) Patent Application Publication
(10) Pub. No.: US 2010/0191302 A1
(43) Pub. Date: Jul. 29, 2010

(54) GASTROINTESTINAL ELECTRICAL STIMULATION FOR THE TREATMENT OF VISCERAL PAIN

(75) Inventors: Jiande Chen, Houston, TX (US); Pankaj Jay Pasricha, Houston, TX (US)

Correspondence Address: WINSTEAD PC, P.O. BOX 50784, DALLAS, TX, 75201, US

(73) Assignee: Board of Regents, University of Texas System

(21) Appl. No.: 12/517,743

(22) PCT Filed: Nov. 30, 2007
(86) PCT No.: PCT/US07/86190

§ 371 Date: Mar. 15, 2010

Related U.S. Application Data
(60) Provisional application No. 60/868,121, filed on Dec. 1, 2006.

(51) Int. Cl. A61N 1/36

(52) U.S. Cl. 607/40

(57) ABSTRACT

The invention is a method of treatment for reducing visceral pain by administering in an individual in need thereof gastrointestinal electrical stimulation in repetitive trains of short pulses, where the administration of gastrointestinal electrical stimulation reduces visceral pain in the individual. Also, provided is a method of treating gastrointestinal sensory dysfunction. Further, this invention provides methods for modulating sympathetic nervous system for the treatment of visceral pain by administering in an individual in need thereof repetitive trains of short pulse electrical stimulation of the sympathetic nerves, where the electrical stimulation provided is effective in reducing visceral pain.

### BACKGROUND OF THE INVENTION

Visceral pain is a common symptom associated with acute gastritis, chronic gastritis, peptic ulcer as well as a number of functional gastrointestinal disorders including IBS, dyspepsia, and GERD. Acute gastritis, an inflammation of the inner lining, is characterized by severe pain in the epigastrium, nausea, vomiting etc. If there is an infection involved there will also be diarrhea, fever, etc. (acute gastroenteritis). Chronic gastritis may follow acute attacks of gastritis or associated with a deficiency of gastric juices, malnutrition, congestive heart failure or uremia, etc. Clinical manifestations are usually distress or pain in the epigastrium, loss of appetite, and abdominal distention, or symptoms that resemble those of peptic ulcer. The patient with a peptic ulcer complains of a cramp like sensation in the epigastrium. Functional gastrointestinal diseases are characterized by altered motility, sensitivity and secretion as well as having a psychological (usually subconscious) overlay as well. IBS, is a chronic condition, and is accompanied by gastric pain, bloating and altered bowel function. Functional dyspepsia is a highly prevalent symptom complex and a heterogenous disorder. Symptomatic improvement of patients with functional dyspepsia after drug therapy is often incomplete and obtained in not more than 60% of patients. Gastroesophageal reflux disease (GERD) is a condition that is associated with the reflux of gastric contents to the esophagus through the lower esophageal sphincter. GERD is characterized by symptoms of gastric pain, heartburn, bloating, epigastric pain, early satiety, nausea, regurgitation, and vomiting.

Current therapies for visceral pain include OTC or prescription products or a combination of both. The presently prescribed medications lose their efficacy value. Various reasons for this loss of efficacy have been postulated. Some of them include a development of tolerance, intolerability of accompanying adverse effects, relief of the motility component but not the other symptoms and signs such as visceral pain and bloating etc.

A need continues to exist for additional feasible and suitable means to treat visceral pain. Likewise, a need continues to exist for additional feasible and suitable means to treat other gastrointestinal tract disorders.

Throughout this application various publications are referenced. The disclosures of each of these publications in their entireties are hereby incorporated by reference in this application.

### SUMMARY OF THE INVENTION

Presented herein are methods for reducing visceral pain. In various embodiments, this method involves administering gastrointestinal electrical stimulation, in repetitive trains of short pulses, to an individual experiencing visceral pain, wherein the administration of gastrointestinal electrical stimulation reduces gastrointestinal pain in the individual.

Also provided herein are methods of modulating spinal afferent neurons for the treatment of visceral pain. Specifically, such methods involve administering to an individual experiencing gastrointestinal pain, repetitive trains of short pulse of gastrointestinal electrical stimulation, wherein such modulation of the spinal afferent neurons is effective in reducing visceral pain.

Additionally, provided herein are methods of treating gastrointestinal sensory dysfunction.

The details of one or more embodiments of the invention are set forth in the accompanying drawings and the description below. Other features, objects, and advantages of the invention will be apparent from the description and drawings, and from the claims.

### BRIEF DESCRIPTION OF THE DRAWINGS

The foregoing and other features and aspects of the present disclosure will be best understood with reference to the following detailed description of specific embodiments of the disclosure, when read in conjunction with the accompanying drawings, wherein:

FIG. 1 shows Behavioral (left) and EMG (right) response to gastric distention before and after electrical stimulation of the stomach, using “dense disperse” waveform at 6 volts (*P<0.05)

FIG. 2 depicts the EMG responses to GES using 100 Hz frequency at 6 v. (*P<0.05).

### DETAILED DESCRIPTION OF THE INVENTION

As used herein, the “gastrointestinal tract” (GI tract) refers to the “gut” or the “alimentary canal” that is a continuous, coiled, hollow, muscular tube that winds through the ventral body cavity. It is open to the external environment at both ends. In a human, its organs (gastrointestinal organs) generally include the mouth, pharynx, esophagus, stomach, small intestine (duodenum, jejunum, and ileum), and large intestine (cecum, appendix, colon, rectum, and anal canal). The large intestine leads to the terminal opening, or anus.

The “gastrointestinal wall” refers to the continuous, coiled, hollow, muscular tube that is the gastrointestinal tract. The wall generally defines the center (lumen) of the GI tract (the hollow portion of the tube). The wall has a thickness defining an interior wall adjacent to the center of the GI tract and an exterior wall.

As used herein, “gastrointestinal action” refers to any GI actions. Thus, gastrointestinal action includes, for example, gastrointestinal electrical activity, gastrointestinal contractile activity (such as stomach contractile activity), gastrointestinal motility, gastric emptying, gastrointestinal pressure, gastrointestinal impedance, and afferent nerve activity (including vagal nerve, sympathetic nerves, and spinal nerves).

“Visceral pain” refers to pain or discomfort that is centered in the upper abdomen and/or the lower abdomen, for example, pain associated with dyspepsia or pain due to irritable bowel syndrome. In one embodiment, the visceral pain is caused by distention or other noxious stimulation of a gastrointestinal organ.

“Reducing” visceral pain refers to reducing or eliminating one or more of the symptoms of visceral pain. Methods of measuring the reduction of visceral pain in a non-human subject include measuring a number of behavioral responses to visceral pain before and after gastrointestinal electrical stimulation is provided. In animals the responses measured include rapid breathing, nausea, vomiting, burping, licking lips. In a human subject, the reduction and/or elimination of symptom of visceral pain is measured by evaluation of the subject by, for example verbal expression of intensity of pain on a scale such as 0-10.

Although not meaning to be bound by theory, gastrointestinal pain of a subject is largely mediated via the sympathetic (spinal cord) pathway. Gastrointestinal electrical stimulation, as used in the present invention, alters sympathetic nerves, such as the spinal afferent neurons. Accordingly, gastrointestinal electrical stimulation treats or reduces pain of a subject by blocking the sympathetic pathway of the subject.

A subject refers to an animal, including a human, subject. For non-human animal subjects, the particular structure of the GI tract may differ from that of a human. For such non-human animal subjects, the gastrointestinal tract, as used herein, refers to that non-human animal's known GI tract and GI organs. It is understood that the first step of the present invention includes selecting a subject which would benefit from the method of the subject, such as, for example, selecting a subject who is suffering from gastrointestinal pain.

An “optimum level” refers to a pre-determined target, which is determined based on the desired outcome. For example, in GES (see below), the definition of optimization is based on an optimal combination of efficacy, safety and feasibility. That is, the optimal GES settings are those that result in a significant reduction in pain (efficacy) but do not induce undesired symptoms, such as nausea or vomiting (safety) with minimal energy (maximally feasible for an implantable device). Iterative adjustments of stimulation parameters are made to achieve this result. For any particular gastrointestinal action, an “optimum level” or desirable level can be determined by monitoring the appropriate GI action. As another example, an appropriate amount of GI pressure at the esophageal sphincter can be determined which prevents reflex of stomach juices into the esophagus, while still allowing the passage of food items into the stomach. With this predetermined “optimum level”, a stimulatory electrode can be established with a sensor to maintain this optimum level. The optimum level is thus fact and subject specific, but readily determinable with routine experimentation, taking into account the goal of an optimal combination of efficacy, safety and feasibility.

A “stimulatory electrode” refers to a conductor of electricity through which current enters a medium (a subject), whereas a “sensor” refers to a conductor of electricity through which current leaves a medium (a subject). Typically, for gastrointestinal uses, the stimulatory electrodes and sensors are constructed of teflon-insulated wires such as are used for cardiac pacing wires. The stimulatory electrode is electrically connected (i.e., conductively connected) to a source of electrical current (often referred to as a pacemaker where a set pattern of electrical current is delivered), and the sensor is electrically connected to a device for determining the level of electrical current “sensed” by the sensor (an electrical recorder, for example). The stimulatory electrode is thus used to “generate” electrical current and the sensor is thus used to “detect” electrical current. Note that the stimulatory electrode can be used to “generate” electrical current, which is itself a defined “gastrointestinal action”, but the generation of electrical current can also produce other gastrointestinal actions (such as, for example, stomach contraction or esophageal pressure). The language “generating” GI action is thus intended to cover both concepts, i.e. the generation of the initial electrical current and the ultimate gastrointestinal action which is “generated” as a result of the current (i.e. the contraction or pressure).

“Operatively connected” is used herein to refer to the connection between the stimulatory electrode and the sensor, and indicates that the operation of one is connected to the operation of the other. In particular, the sensor connects to a device which determines the level of electrical current sensed by the sensor. A representation of that level is then fed to the source of electrical current that is electrically connected to the stimulatory electrode. The source of electrical current is provided with a programmable computer circuit that enables the level from the sensor to determine, or dictate, the operation of the source (i.e., electrical current is generated by the source and fed through the stimulatory electrode in response to and an in relation to the amount of the level of electrical activity sensed by the sensor). Thus, the “operatively connected” stimulatory electrode and sensor enable the retrograde feedback concept to occur.

“Positioning” a stimulatory electrode or a sensor refers to placement of the stimulatory electrode or sensor on or in a subject. Placement or positioning of stimulatory electrodes can be accomplished by laproscopic, endoscopic or surgical means.

“Periodically” refers to evenly or unevenly spaced time intervals.

“Differs from” refers to a statistically significant variation between two compared values, and therefore does not always require a difference in orders of magnitude. It should be apparent that where small values are compared, statistically significant variations can likewise be very small, and where large values are compared, statistically significant variations can be large. Conversely, “substantially equals” refers to a statistically insignificant variation between two compared values.

“Electrical field stimulation” refers to the generation of an “electrical field”, which indicates that the area of distribution of the electrical current from the stimulation encompasses the entire area between and/or surrounding two or more stimulatory electrodes, and “field” is used to imply that the two or more stimulatory electrodes are positioned at least about three centimeters apart (thus the term “field” to differ from prior stimulations where the two electrodes of a pair are positioned in close proximity to one another and do not generate a “field”).

A “device” refers to any suitable item which can readily be and is desirable to be placed in the GI tract. Such devices can include, for example, stimulatory electrodes and sensors for use in the GES method of the subject invention. Such devices could also include a small balloon to be used to provide pressure within the esophagus or small/large intestine. A small gauge for measurement of pressure could be a device in accordance with the subject invention.

Electrical stimulation refers to an electrical signal, which includes a train of pulses. A train of pulses refers to a method in which the stimulus is composed of repetitive trains of short pulses derived from a combination of two signals, a) a continuous short pulse with high frequency (in the order of 5 to 150 Hz), and b) control signal to turn the pulses on or off, such as “X” seconds on and “Y” seconds off. The addition of “X” and “Y” then determines the frequency of the pulse train. A frequency approximately equal to the physiologic frequency of stimulation will be performed using trains of pulses. The train will be set on for a period of 0.1 s to 5 seconds and set off for a period of 0 to 10 min. The pulses within a train have a frequency of 5 to 150 Hz width of 0.1 to 2 ms and amplitude of 0.1 mA to 10 mA or the corresponding voltages that will produce the described current. The methods of providing electrical field stimulation to a gastrointestinal organ are disclosed in WO/2001/076690 (GASTROINTESTINAL ELECTRICAL STIMULATION) which is hereby incorporated by reference herein. A discussion of trains of short pulse electrical stimulation is provided in Zhang et al., Current treatments of Gastroenterol. 9: 351-360 (2006), which is hereby incorporated by reference herein. Gastrointestinal electrical stimulation, is used herein to alter sympathetic nerves, such as the spinal afferent neurons for the treatment of pain.

The sympathetic nerve fibers, along with many of the spinal cord's nerve root fibers, and the cranial nerves that innervate tissue in the thoracic and abdominal cavities are sometimes referred to as the autonomic, or vegetative, nervous system. The sympathetic, spinal, and cranial nerves all have couplings to the central nervous system, generally in the primitive regions of the brain, however, these components have direct effects over many regions of the brain, including the frontal cortex, thalamus, hypothalamus, hippocampus, and cerebellum. The central components of the spinal cord and the sympathetic nerve chain extend into the periphery of the autonomic nervous system from their cranial base to the coccyx, essentially passing down the entire spinal column, including the cervical, thoracic and lumbar regions. The sympathetic chain extends on the anterior of the column, while the spinal cord components pass through the spinal canal. The cranial nerves, the one most innervating of the rest of the body being the vagus nerve, passes through the dura mater into the neck, and then along the carotid and into the thoracic and abdominal cavities, generally following structures like the esophagus, the aorta, and the stomach wall.

Gastrointestinal functions are controlled by various cranial nerves that traverse portions of the human body. The rich sensory innervation of the gastrointestinal tract comprises intrinsic sensory neurons contained entirely within the gastrointestinal wall, intestinofugal fibers that project to prevertebral ganglia, and vagal and spinal afferents that project into the central nervous system. This dense intrinsic sensory innervation serves to control motor and secretory functions in response to the local environment in the gastrointestinal wall or lumen. Afferent fibers convey a vast amount of sensory information to the brainstem and spinal cord, but the nature of this information is different for the vagal and spinal pathways. These afferents are sensitive to both mechanical and chemical stimuli. Vagal afferents convey predominantly physiological information, whereas spinal afferents are able to encode noxius stimuli. The spinal afferents encode both physiological and supraphysiological levels of interstinal pressure and therefore form the main pathway for mediating pain perception. Spinal afferents have a more promiscuous type of chemosensitivity as opposed to specific chemical sensitivity that may be involved in signal transduction of vagal afferent system. Vagal mechanosensitive fibers, on the other hand, extend into the muscle where, together with intraganglionic lamina endings, they form a transduction site for mechanosensitivity. Spinal afferents respond to distention over a wide dynamic range extending from physiological to noxious levels. These spinal endings contribute to signaling visceral pain through some intensity code that recognizes extreme levels of distention or contraction.

The peripheral terminals of vagal and spinal afferents are localized within the gastrointestinal wall using antegrade tracing techniques. Their location in mucosal layers, muscle, and in the serosal and mesenteric attachments are consistent with their responses to stimuli acting at these different sites within the gastrointestinal wall. The vagal afferents play a pivotal role in gastric chemonociceotion, particularly in the pain reaction to gastric acid challenge. The acid sensitivity of vagal afferent system is upregulated by endogenous acid secretion, cytokines, gastric inflammation and gastric ulceration. Hence, chemosensitive vagal nerve fibers are involved in the upper abdominal hyperalgesia associated with acid-related disorders including functional dyspepsia.

Electrical stimulation of the gastrointestinal tract has been proposed to treat motility related disorders and other gastrointestinal diseases. The electrical stimulation has been proposed in a number of forms, such as, e.g., pacing, electrical contractile stimulation or other stimulation, e.g., to treat nausea or obesity. Electrical pacing of the gastrointestinal tract is generally defined as a periodic electrical stimulation that captures and/or controls the frequency of the pacesetter potential or slow wave activity of the intestinal organ (including in a retrograde direction). Electrical contractile stimulation generally refers to stimulation that directly causes or results in muscular contraction associated with the gastrointestinal tract. Gastric electrical stimulation (GES) has been suggested as a therapy for morbid obesity and gastrointestinal motility disorders. There have been a number of reports on Gastric electrical stimulation for the treatment of gastrointestinal motility disorders in both dogs and humans (U.S. Pat. Nos. 5,423,872, 5,690,691, and 5,836,994; PCT International Publication No. WO 99/30776; Bellahsene et al. 1992; Mintchev et al. 1998; Mintchev et al. 1999; Mintchev et al. 2000; Chen et al. 1998; Chen et al. 1995c). These disorders are characterized by poor contractility and delayed emptying and the aim of electrical stimulation in this setting is to normalize the underlying electrical rhythm and improve these parameters. In general, this is done by antegrade or forward gastric (or intestinal) stimulation. Previous work on antegrade gastrointestinal stimulation has been focused on its effects on gastric myoelectrical activity, gastric motility, and gastric emptying, (Lin et al. 1998; Eagon and Kelly 1993; Hocking et al. 1992; Lin et al. 2000a; McCallum et al. 1998; Miedema et al. 1992; Qian et al. 1999; Abo et al. 2000; Bellahsene et al. 1992). Hence, provided herein is a method of treatment for reducing visceral pain of a subject by administering to an individual in need thereof repetitive trains of short pulse gastrointestinal electrical stimulation, effective in reducing the visceral pain of the subject. Specifically, the electrical stimulation provided in repetitive trains of pulses are set on for a range of period between about 0.1 seconds to less than or equal to 5 seconds and set off for a range of period between about 0 to less than or equal to 10 minutes. Additionally, the repetitive trains of pulses are administered in the range between about 1 Hz. to less than or equal to 150 Hz. Moreover, the repetitive trains of pulses have a pulse width in the range between about 0.1 ms to less than or equal to 2.0 ms. In general, the repetitive trains of pulses have an amplitude in the range between about 0.1 mA to less than or equal to 20.0 mA. Specifically, the gastrointestinal electrical stimulation is provided by stimulatory electrodes. Moreover, the stimulatory electrodes may be placed in the stomach, small intestines, colon or anorectum. Further, the placement of the electrodes may be accomplished by laproscopic, endoscopic or surgical means. Generally, the visceral pain is due to gastric distention, functional dyspepsia, constipation, diarrhea, fecal incontinence, pseudo-obstruction, Gastroesophageal Reflux Disease, irritable bowel syndrome, reduced gastric accommodation, gastroenteritis, enhanced visceral sensitivity, indigestion, gastroesophageal reflux, Helicobacter pylori infection, or gastroparesis.

Also provided are methods of modulating the activity of the spinal afferent neurons by administering to an individual in need thereof repetitive trains of short pulse gastrointestinal electrical stimulation, where the modulation of the activity of the spinal afferent neurons is effective in reducing visceral pain.

Additionally, provided is a method of treating gastrointestinal sensory dysfunction by administering in an individual in need thereof repetitive trains of short pulse gastrointestinal electrical stimulation, where the gastrointestinal electrical stimulation is effective in treating gastrointestinal sensory dysfunction.

In one embodiment the electrical stimulation is single channel and in other alternative embodiments the electrical stimulation is dual channel or three channel.

The present invention also encompasses enhancing the therapeutic effects of other therapies, such as methods and systems working in conjunction with a pharmaceutical agent or other therapies to augment, enhance, improve, or facilitate other therapies (adjunctive therapies) as well as reducing/minimize and counteracting side effects, complications and adverse reactions for any therapies involved in treating the above-mentioned medical conditions.

### Example 1

### Effect of Gastric Electrical Stimulation on Gastric Distention-Induced Vomiting and Behavior Changes in Dogs.

Seven dogs were involved in this study. The experiment was performed in 2 sessions on separate days in a randomized order control and GES.

A gastric balloon connected to a barostat device was inserted into the dog's stomach from a gastric cannula. The stomach was distended using the barostat via the intragastric balloon by gradually increasing the pressure until the maximum tolerance by the animals. The distention was then maintained for 5 min and the signs were recorded and scored. The procedure of the GES was the same except that GES was performed using the following parameters: 0.1 s on, 5 s off, 14 Hz, 330 μs and 5 mA.

The results are summarized in the following table.

| | Pressure | Sign score | | Sign score | |
|---|---|---|---|---|---|
| Dog number | (mmHg) | (Non-GES) | | (GES) | |
| 7114 | 38 | 40 | (N) | 39 | (N) |
| 7673 | 26 | 36 | (V) | 23 | (N) |
| 7357 | 24 | 54 | (N) | 38 | (V) |
| 7247 | 18 | 25 | (V) | 15 | (N) |
| 7761 | 24 | 37 | (N) | 10 | (N) |
| 7763 | 20 | 61 | (Barking) | 60 | (N) |
| 6362 | 20 | 48 | (V) | 24 | (N) |
| Total | | 301 | | 209 | |

V: Vomiting
NV: Non-Vomiting

It was concluded that GES reduces vomiting and improves gastric distention-induced signs/symptoms in dogs.

### Example 2

### Involvement of the Sympathetic Afferent Pathway in Reducing Gastrointestinal Pain by Gastrointestinal Electrical Stimulation

The effects of different gastrointestinal electrical stimulation parameters on visceral pain induced by acid in rats was assessed using extracellular recordings of the spinal cord afferent neurons. 20 SD male rats (280-350 g) were used in this study. 10 ul of 20% acetic acid were injected into 15 sites in the submucosal layer of the stomach wall to create a visceral hypersensitivity model for gastrointestinal pain.

A behavioral study of visceral sensitivity (discomfort and pain) was assessed based on the measurement of muscle activity (electromyography or EMG) from the animal neck for a 30-min period at baseline, during a 15-min period of stomach distention and a 30-min period after distention at different distension pressures (20, 40, 60, 80 mmHg). Further, to assess the involvement of the sympathetic afferent pathway, T9-T10 spinal cord cell spike activity in response to gastrointestinal distention at different pressures (20, 40, 60 mmHg) was recorded. These recordings were made before GES (baseline), during GES 15 min and after GES. The gastrointestinal electrical stimulation was provided at a frequency ranging from 10-100 Hz, the pulse width was 0.25-0.5 ms, and the train on-time was 0.1 s-3 s on.

The parameter 0.1 s on, 0.4 s off, 100 Hz, 6 mA gastrointestinal electrical stimulation decreased the EMG significantly at gastric distention of 40, 60 and 80 mmHg, compared with baseline (2249.98±596.33 VS 4128.03±889.63; 4501.15±639.13 VS 7271.99±963.29; 6841.03±863.12 VS 13758.13±1769.88) (P<0.05). 30 spinal cord cells in visceral hypersensitivity rats were studied. 8 cells were high threshold cells to GD (gastric distension), 9 cells were low threshold to GD. Compared with baseline, GES increases low-threshold cells total response during GD (487.4±56.3 Vs 340.2±33.8), but decreased high-threshold cells total response during GES (208.8±98.6 Vs 496.5±163.3) (P<0.05).

GES with appropriate parameters reduced gastric distention-induced visceral pain reflected as a decrease in EMG activity and this inhibitory effect may be mediated by the sympathetic afferent pathway reflected as a blockage of pain cells (high threshold spinal cord neurons).

### Example 3

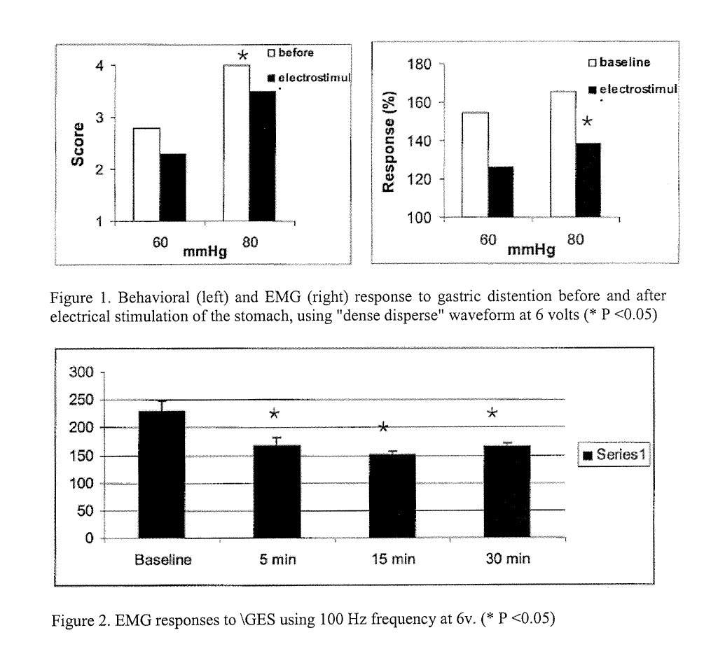

Dyspepsia, or pain or discomfort centered in the upper abdomen, is a common condition accounting for 2-5% of all primary care consults with an estimated prevalence of 25%. Although a variety of pathophysiologic mechanisms have been implicated in the etiology (such as delayed gastric emptying, diminished gastric accommodation, Helicobacter pylori infection, enhanced visceral sensitivity, food intolerance and psychological factors, there is no single unifying hypothesis that can explain the syndrome entirely and no satisfactory pharmacological treatment exists. There is therefore a need for alternative therapies. Gastric electrical stimulation (GES) has additionally been thought to modulate vagal-brain signaling. We hypothesized that GES would also modulate spinal pathways for perception of pain and other sensations and hence have the potential for treating functional dyspepsia. We tested this in rats using a pair of electrodes in the body and stimulated the stomach using at least two different kinds of parameters. Gastric pain was elicited by inflation of a balloon in the stomach and the response measured on a behavioral scale that correlated with pain as well as by a quantitative assessment of the visceromotor reflex using electromyography of the sternocleidomastoid muscle. The results are shown in FIGS. 1 and 2.

Although preferred embodiments have been depicted and described in detail herein, it will be apparent to those skilled in the relevant art that various modifications, additions, substitutions and the like can be made without departing from the spirit of the invention and these are therefore considered to be within the scope of the invention as defined in the claims which follow. Any printed documents referred to herein are hereby incorporated by reference as if the documents were presented in their entirety herein.

What is claimed is:

1. Use of gastrointestinal electrical stimulation for the treatment of visceral pain comprising administration of repetitive trains of short pulse gastrointestinal electrical stimulation effective in treating visceral pain.

2. The use of claim 1, wherein said repetitive trains of pulses are set on for a range of period between about 0.1 seconds to less than or equal to 5 seconds and set off for a range of period between about 0 to less than or equal to 10 minutes.

3. The use of claim 1, wherein said repetitive trains of pulses are administered in the range between about 1 Hz. to less than or equal to 150 Hz.

4. The use of claim 1, wherein said repetitive trains of pulses have a pulse width in the range between about 0.1 ms to less than or equal to 2.0 ms.

5. The use of claim 1, wherein said repetitive trains of pulses have an amplitude in the range between about 0.1 mA to less than or equal to 20.0 mA.

6. The use of claim 1, wherein said gastrointestinal electrical stimulation is provided by stimulatory electrodes placed in the stomach, small intestines, colon or anorectum.

7. The use of claim 6, wherein said electrodes are placed by laproscopic, endoscopic or surgical means.

8. The use of claim 1, wherein said gastrointestinal stimulation is effective in modulating the activity of the spinal afferent neurons

9. The use of claim 1, wherein said visceral pain is due to gastric distention, functional dyspepsia, constipation, diarrhea, fecal incontinence, pseudo-obstruction, Gastroesophageal Reflux Disease, irritable bowel syndrome, reduced gastric accommodation, gastroenteritis, enhanced visceral sensitivity, indigestion, gastroesophageal reflux, Helicobacter pylori infection, or gastroparesis.

10. Use of gastrointestinal electrical stimulation for the treatment gastrointestinal sensory dysfunction comprising administration of repetitive trains of short pulse gastrointestinal electrical stimulation, wherein said administration is effective in the treatment of gastrointestinal sensory dysfunction.

11. The use of claim 10, wherein said gastrointestinal sensory dysfunction causes visceral pain.

12. The use of claim 10, wherein said repetitive trains of pulses are set on for a range of period between about 0.1 seconds to less than or equal to 5 seconds and set off for a range of period between about 0 to less than or equal to 10 minutes.

13. The use of claim 10, wherein said repetitive trains of pulses are administered in the range between about 1 Hz. to less than or equal to 150 Hz.

14. The use of claim 10, wherein said repetitive trains of pulses have a pulse width in the range between about 0.1 ms to less than or equal to 2.0 ms.

15. The use of claim 10, wherein said repetitive trains of pulses have an amplitude in the range between about 0.1 mA to less than or equal to 20.0 mA.

16. The use of claim 10, wherein said gastrointestinal electrical stimulation is provided by stimulatory electrodes placed in the stomach, small intestines, colon or anorectum.

17. The use of claim 16, wherein said electrodes are placed by laproscopic, endoscopic or surgical means.

18. The use of claim 10, wherein said gastrointestinal stimulation is effective in modulating the activity of the spinal afferent neurons

19. The use of claim 11, wherein said visceral pain is due to gastric distention, functional dyspepsia, constipation, diarrhea, fecal incontinence, pseudo-obstruction, Gastroesophageal Reflux Disease, irritable bowel syndrome, reduced gastric accommodation, gastroenteritis, enhanced visceral sensitivity, indigestion, gastroesophageal reflux, Helicobacter pylori infection, or gastroparesis.

20. Use of gastrointestinal electrical stimulation for the modulation of spinal afferent neurons comprising administration of repetitive trains of short pulse gastrointestinal electrical stimulation wherein said modulation of the spinal afferent neurons is effective in reducing visceral pain.

21. The use of claim 20, wherein said visceral pain is due to gastric distention, functional dyspepsia, constipation, diarrhea, fecal incontinence, pseudo-obstruction, Gastroesophageal Reflux Disease, irritable bowel syndrome, reduced gastric accommodation, gastroenteritis, enhanced visceral sensitivity, indigestion, gastroesophageal reflux, Helicobacter pylori infection, or gastroparesis.

22. A method of treatment for reducing visceral pain comprising:
administering to an individual in need thereof gastrointestinal electrical stimulation in repetitive trains of short pulses, wherein said administration of gastrointestinal electrical stimulation reduces gastrointestinal pain in said individual.

23. A method of treating gastrointestinal sensory dysfunction comprising:
administering in an individual in need thereof repetitive trains of short pulse gastrointestinal electrical stimulation, wherein said gastrointestinal electrical stimulation is effective in treating gastrointestinal sensory dysfunction.

24. A method of modulating the activity of the spinal afferent neurons comprising:
administering in an individual in need thereof repetitive trains of short pulse gastrointestinal electrical stimulation, wherein said modulation of the spinal afferent neurons is effective in reducing visceral pain.
